(19) United States
(12) Patent Application Publication
(10) Pub. No.: US 2004/0152695 A1
(43) Pub. Date: Aug. 5, 2004

(54) Pharmaceutical composition of 1-(3-hydroxy-4-methoxyphenyl)-4-methyl-5-ethyl-7,8-dimethoxy-5H-2,3-benzodiazepine and uses thereof

(75) Inventors: Herbert W. Harris, Merion, PA (US); Steven M. Leventer, Langhorne, PA (US); Robert F. Kucharik, Glenmoore, PA (US)

Correspondence Address: DRINKER BIDDLE & REATH, ONE LOGAN SQUARE, 18TH AND CHERRY STREETS, PHILADELPHIA, PA, 19103-6996, US

(73) Assignee: Vela Pharmaceuticals, Inc., Lawrenceville, NJ

(21) Appl. No.: 10/728,179

(22) Filed: Dec. 3, 2003

Related U.S. Application Data
(60) Provisional application No. 60/430,770, filed on Dec. 3, 2002.

(51) Int. Cl. A61K 31/5513

(52) U.S. Cl. 514/221

(57) ABSTRACT

Pharmaceutical compositions comprise 1-(3-hydroxy-4-methoxyphenyl)-4-methyl-5-ethyl-7,8-dimethoxy-5H-2,3-benzodiazepine, or a pharmaceutically acceptable salt thereof. The compositions are used for treating, preventing or delaying the onset of disorders mediated by LTB₄ or TXA₂.

### CROSS-REFERENCE TO RELATED APPLICATION

This application claims the benefit of copending U.S. Provisional Application Serial No. 60/430,770, filed Dec. 3, 2002, the entire disclosure of which is herein incorporated by reference.

### FIELD OF THE INVENTION

The present invention relates to pharmaceutical compositions comprising 1-(3-hydroxy-4-methoxyphenyl)-4-methyl-5-ethyl-7,8-dimethoxy-5H-2,3-benzodiazepine, and to uses of such compounds in methods of treatment.

### BACKGROUND OF THE INVENTION

2,3-Benzodiazepines

Certain 2,3-benzodiazepines have been explored extensively for their potent CNS modulating activity. Compounds such as tofisopam (Grandaxin®)(structure shown below, with the atom numbering system indicated), girisopam, and norisopam have demonstrated substantial anxiolytic and antipsychotic activity.

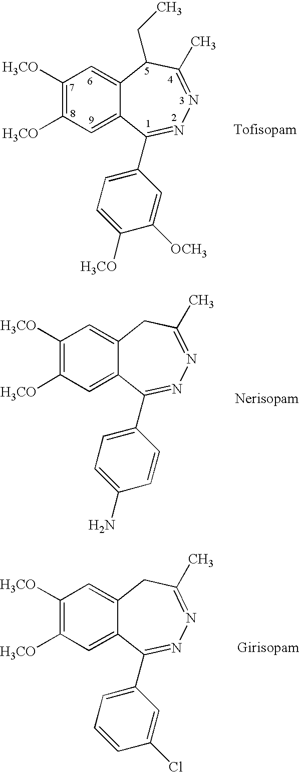

Tofisopam has been shown in humans to have an activity profile that is significantly different from that of widely used 1,4-benzodiazepine (BZ) anxiolytics such as diazepam (Valium®) and chlordiazepepoxide (Librium®). The 1,4-benzodiazepines, in addition to having sedative-hypnotic activity, also possess muscle relaxant and anticonvulsant properties which, though therapeutically useful in some disease states, are nonetheless potentially untoward side effects. Thus the 1,4-benzodiazepines, though safe when administered alone, may be dangerous in combination with other CNS drugs, including alcohol.

Tofisopam, in contrast, is a non-sedative anxiolytic that has no appreciable sedative, muscle relaxant or anticonvulsant properties (Horvath et al., Progress in Neurobiology, 60 (2000), 309-342). In clinical studies, tofisopam improved rather than impaired psychomotor performance and showed no interaction with ethanol (Id.). These observations comport with data that show that tofisopam does not interact with central BZ receptors and binds only weakly to peripheral BZ receptors.

Other 2,3-benzodiazepines that are structurally similar to tofisopam have been investigated and shown to have varying activity profiles. For example, GYKI-52466 and GYKI-53655 (structures shown below) act as noncompetitive glutamate antagonists at the AMPA (α-amino-3-hydroxy-5-methyl-4-isoxazolepropionic acid) site, and have demonstrated neuroprotective, muscle relaxant and anticonvulsant activity (Id.). Another group of 2,3-benzodiazepines that have been investigated are represented by the compound GYKI-52895, and show activity as selective dopamine uptake inhibitors with potential use in antidepressant and anti-Parkinsonism therapy.

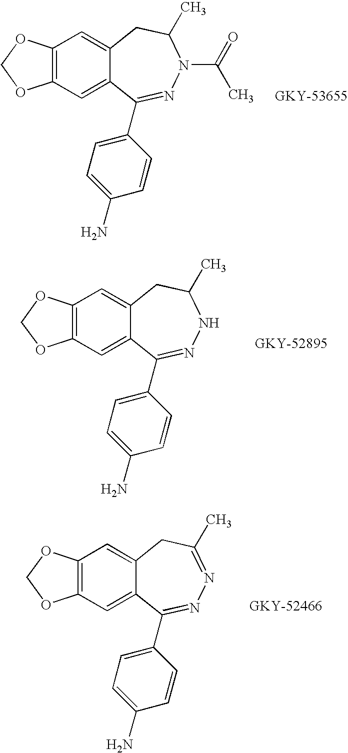

Tofisopam is a racemic mixture of (R)- and (S)-enantiomers. This is due to the asymmetric carbon, i.e., a carbon with four different groups attached, at the 5-position of the benzodiazepine ring.

The molecular structure and conformational properties of tofisopam have been determined by NMR, CD and x-ray crystallography (Visy et al., Chirality 1:271-275 (1989)). The 2,3-diazepine ring exists as two different conformers. The major conformers, (+)R and (−)S have the 5-ethyl group in a quasi-equatorial position, while in the minor conformers, (−)R and (+)S, the 5-ethyl group is positioned quasi-axially. Thus, racemic tofisopam may exist as four molecular species, i.e., two enantiomers, each of which exists in two conformations. The sign of the optical rotation is reversed upon inversion of the diazepine ring from one conformer to the other. In crystal form, tofisopam exists only as the major conformations, with dextrorotatory tofisopam being of the (R) absolute configuration. (Toth et al., J. Heterocyclic Chem., 20:709-713 (1983); Fogassy et al., Bioorganic Heterocycles, Van der Plas, H. C., Ötvös, L, Simongi, M., eds. Budapest Amsterdam: Akademia; Kiado-Elsevier, 229:233 (1984)).

Differential binding of the (+) and (−) conformers of 2,3-benzodiazepines generally, has been reported for tofisopam in binding studies with human albumin (Simongi et al. Biochem. Pharm., 32(12), 1917-1920, 1983). The (+) and (−) conformers of tofisopam have also been reported as existing in an equilibrium (Zsila et al., Journal of Liquid Chromatography & Related Technologies, 22(5), 713-719, 1999; and references therein).

The optically pure (R)-enantiomer of tofisopam (R)-1-(3,4-dimethoxyphenyl)-4-methyl-5-ethyl-7,8-dimethoxy-5H-2,3-benzodiazepine) has been isolated and shown to possess the nonsedative anxiolytic activity of the racemic mixture. See U.S. Pat. No. 6,080,736; the entire disclosure of which is incorporated herein by reference.

Metabolism of Tofisopam

Tofisopam is metabolized in human, rat, dog, monkey and rabbit to one or more of six major metabolites, depending on the host species:

| Compound # | Compound Name |
|---|---|
| 1 | 1-(3,4-dimethoxyphenyl)-4-methyl-5-ethyl-7-hydroxy-8- |
| | methoxy-5H-2,3-benzodiazepine |
| 2 | 1-(3,4-dimethoxyphenyl)-4-methyl-5-ethyl-7-methoxy-8- |
| | hydroxy-5H-2,3-benzodiazepine |
| 3 | 1-(3-methoxy-4-hydroxyphenyl)-4-methyl-5-ethyl-7,8- |
| | dimethoxy-5H-2,3-benzodiazepine |
| 4 | 1-(3-hydroxy-4-methoxyphenyl)-4-methyl-5-ethyl-7,8- |
| | dimethoxy-5H-2,3-benzodiazepine |
| 5 | 1-(3-methoxy-4-hydroxyphenyl)-4-methyl-5-ethyl-7- |
| | hydroxy-8-methoxy-5H-2,3-benzodiazepine |
| 6 | 1-(3-hydroxy-4-methoxyphenyl)-4-methyl-5-ethyl-7- |
| | hydroxy-8-methoxy-5H-2,3-benzodiazepine |

See Tomori et al., Journal of Chromatography, 241 (1982), p. 89-99.

Of the compounds named above, Compounds 1, 3 and 5 have been identified as metabolites in humans. These compounds have been synthesized and tested in certain pharmacological assays. C. Ito, “Behavioral Pharmacological Study on the Structure Activity Relationship of Benzodiazepine Derivatives: With Particular Reference to the Activity of 2,3-Benzodiazepine,” J. Tokyo Med. College, 39:369-384 (1981)._In an assay of inhibition of aggression in mice, Compound 1 and 3 showed 0% inhibition of aggression and Compound 5 showed a 28.6% inhibition of aggression. In an assay of muricide (mouse killing behavior) in rats, Compound 3 exhibited 0% inhibition of muricide while Compounds 1 and 5 each exhibited a 20% inhibition of muricide. In assays testing for anti-noradrenergic effects, Compound 1 exhibited no effect, while Compounds 3 and 5 demonstrated measurable activity.

Compounds 1, 3, 5 and 6 are also disclosed in U.S. Pat. No. 4,322,346, the entire disclosure of which is incorporated herein by reference. Compound 3 is reported therein to demonstrate narcosis-potentiating activity in mice.

Leukotriene B₄ (LTB₄)

Leukotrienes, along with prostaglandins and thromboxanes, are products of arachidonic acid metabolism. LTB₄ is produced by leukocytes, particularly macrophage and monocytes upon activation by immune complexes, phagocytosis or other stimuli. LTB₄ is a potent chemotactic agent that stimulates neutrophil and macrophage migration (chemotaxis) to sites of inflammation. The structure of LTB₄ is shown below.

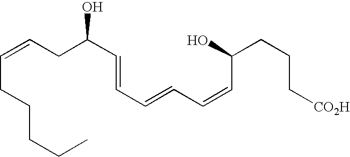

The known pathophysiological responses of LTB₄ include: induction of potent neutrophil chemotactic activity, promotion of adhesion of polymorphonuclear leukocytes (PMN) to vasculature, increase in vascular permeability, stimulation of the release of lysosomal enzymes, by PMN. The pro-inflammatory action of LTB₄ has been demonstrated in vivo, wherein topical LTB₄ on human skin promotes the infiltration of PMN and other inflammatory cells. Intradermal injection of LTB₄ induces accumulation of neutrophils at the injection site. Intravenous injection of LTB₄ causes rapid but transient neutropenia. See Kingsbury et al., J. Med. Chem., 1993, 36, 3308-3320; and references cited therein, the entire disclosures of which are incorporated herein by reference.

In addition, the presence of physiologically relevant LTB₄ concentration at inflammatory sites has been associated with, for example, disease states such as psoriasis, asthma and active gout; in colonic mucosa associated with inflammatory bowel disease; in synovial fluid from patients with active rheumatoid arthritis; and in reperfusion injury. All of these observations together support the involvement of LTB₄ in human inflammatory disease (Kingsbury et al, and Griffeths et al., Proc. Natl. Acad. Sci. Vol. 92, pp517-521, January 1995; and references cited therein.).

Inflammatory Disorders

Crohn's disease and ulcerative colitis, collectively referred to as inflammatory bowel disease (IBD), are chronic recurrent inflammatory diseases of unclear etiology, affecting the small intestine and colon. Inflammatory bowel disease (IBD) can involve either or both the small and large bowel. These disorders fall into the category of “idiopathic” inflammatory bowel disease because the etiology for them is unknown.

Pathologic findings are generally not specific, although they may suggest a particular form of IBD. “Active” IBD is characterized by acute inflammation. “Chronic” IBD is characterized by architectural changes of crypt distortion and scarring. The term “crypt” refers to a deep pit that protrudes down into the connective tissue surrounding the small intestine. Crypt abscesses (active IBD characterized by the presence of neutrophils in crypt lumens) can occur in many forms of IBD, not just ulcerative colitis. Under normal conditions the epithelium at the base of the crypt is the site of stem cell proliferation and the differentiated cells move upwards and are shed 3-5 days later at the tips of the villi. This normal process, necessary for proper bowel function, is interrupted by IBD

Ulcerative colitis (UC) involves the colon as a diffuse mucosal disease with distal predominance. The rectum is virtually always involved, and additional portions of colon may be involved extending proximally from the rectum in a continuous pattern. Most often ulcerative colitis occurs in young people 15 to 40 years of age. Ulcerative colitis occurs only in the inner lining of the colon (large intestine) or rectum. When it is localized in the rectum, it is called “proctitis”.

Crohn's Disease is a chronic inflammatory disease that has periods of remission (time when person feels well) and relapse (when a person feels ill). Crohn's disease is an inflammation and ulceration process that occurs in the deep layers of the intestinal wall. The most common areas affected are the lower part of the small intestine, called the ileum, and the first part of the colon. This type of Crohn's disease is called ileocolitis. Crohn's disease can infrequently affect any part of the upper gastrointestinal tract. Aphthous ulcers, which are similar to cold sores, are common. Ulcers can also occur in the esophagus, stomach and duodenum.

Therapy for IBD has historically included administration of corticosteroids. However drawbacks of long term corticosteroid therapy include masking (or induction) of intestinal perforation, osteonecrosis and metabolic bone disease. Additional problems relate to development of corticosteroid dependency (Habnauer, New England Journal of Medicine, 334(13), p 841-848, 1996). Aminosalicylates such as sulfasalazine and mesalamine have been used to treat mild or moderately active ulcerative colitis and Crohn's Disease, and to maintain remission (Id at 843). Immunomodulatory drugs such as azathioprine and mercaptopurine have been used in long term treatment for patients with IBD. Common complications with both of these drugs include pancreatitis, which occurs with an incidence of 3-15% of patients, and bone marrow suppression, which requires regular monitoring. More potent immunosuppressive drugs such as cyclosporine and methotrexate have been employed, but toxicity of these drugs limits their use to specific situations of refractory disease states. Other therapeutic approaches include antibiotic therapy and nutritional therapy. Often, therapy involves a combination of the above-described drug therapies in addition to surgical resection of the bowel.

There is no cure for IBD. Ultimately, the chronic and progressive nature of IBD demands a long-term treatment that maximizes the local antiinflammatory effect while minimizing the global systemic effect on the immune system.

Chronic inflammatory disorders such as Crohn's Disease typically demonstrate periods of remission between intervals when the inflammatory is active and requires acute treatment. This is an example of a circumstance wherein it is known beforehand that an individual will develop, or is likely to develop an inflammatory disorder.

Another chronic inflammatory condition believed to be mediated by LTB₄ is psoriasis. Psoriasis is a chronic, recurrent, papulosquamous plaque on areas of trauma such as the elbow, knee or scalp, though it may appear elsewhere on the skin. Psoriasis may coexist with lupus erythematosis in some individuals. Current treatments include topical administration of psoralens. “Psoralens” refers to a group of substances found in many different plants, especially psoralea corylifolia. Psoralens interact with nucleic acids and are also used as research tools. Psoriasis is also treated by long-wave ultraviolet radiation. Neither treatment cures or prevents recurrence of psoriasis symptoms.

Another chronic inflammatory disorder believed to be mediated by LTB₄ is rheumatoid arthritis, which is an autoimmune disease of the joints. Rheumatoid arthritis is characterized by the following criteria 1-7, wherein criteria 1-4 are present for more than 6 weeks: (1) morning stiffness in and around joints lasting at least one hour before maximum improvement; (2) soft tissue swelling (arthritis) of three or more joints observed by a physician; (3) swelling (arthritis) of the proximal interphalangeal, metacarpal phalangeal, or wrist joints; (4) symmetric swelling; (5) rheumatoid nodules, i.e., a granulomatous lesion characterized by central necrosis encircled by a palisade of monocytes and an exterior mantle of lymphocytic infiltrate. These lesions present as subcutaneous nodules, especially at pressure points such as the elbow in individuals with rheumatoid arthritis or other rheumatoid disorders; (6) presence of rheumatoid factors, i.e., an autoantibody in the serum of individuals with rheumatoid arthritis; and (7) roentgenographic erosions, i.e., joint lesions visible on an X-ray.

Rheumatoid arthritis is a chronic disorder for which there is no known cure. The major goals of treatment of rheumatoid arthritis are to reduce pain and discomfort, prevent deformities and loss of joint function, and maintain a productive and active life. Inflammation must be suppressed and mechanical and structural abnormalities corrected or compensated by assistive devices. Treatment options include reduction of joint stress, physical and occupational therapy, drug therapy, and surgical intervention.

There are three general classes of drugs commonly used in the treatment of rheumatoid arthritis: non-steroidal anti-inflammatory agents (NSAID's), corticosteroids, and remittive agents or disease modifying anti-rheumatic drugs (DMARD's). NSAID's and corticosteroids have a short onset of action while DMARD's can take several weeks or months to demonstrate a clinical effect. DMARD's include leflunomide (Arava™), etanercept (Enbrel™), infliximab (Remicade™), antimalarials, methotrexate, gold salts, sulfasalazine, d-penicillamine, cyclosporin A, cyclophosphamide and azathioprine. Because cartilage damage and bony erosions frequently occur within the first two years, rheumatologists now move more aggressively to a DMARD agent.

Treatment of rheumatoid arthritis by chronic administration of a corticosteroid involves the same side effect profile as discussed regarding IBD above. Chronic administration of NSAID's also produces side effects. The most common toxicity of NSAID's is gastrointestinal disturbance. Because prostaglandins play a role in the regulation of renal blood flow and maintenance of glomerular filtration, NSAID's can impair renal function in certain patients. Weight gain and cushingoid appearance is a frequent problem and source of patient complaints. Recent studies have raised concern over the increased cardiovascular risk and accelerated osteoporosis associated with low dose prednisone particularly at doses above 10 mg daily.

Gout is another inflammatory disorder believed to be mediated by LTB₄. Gout is characterized by a disturbance of uric-acid metabolism occurring chiefly in males. Gout is characterized by painful inflammation of the joints, especially of the feet and hands, and arthritic attacks resulting from elevated levels of uric acid in the blood and the deposition of urate crystals around the joints. The condition can become chronic and result in deformity.

Gout can present another circumstance wherein it is known beforehand that an individual will or is likely to develop an inflammatory disorder. In the instance of patients undergoing radiotherapy or chemotherapy, the individual may experience a dramatic rise in serum uric acid levels associated with lysis of the tumor mass. Such large increases in uric acid can deposit urate crystals in synovial fluid of joints thereby causing the inflammatory disorder, gout. When such a rise in serum uric acid levels is known to be likely, prophylaxis with an LTB₄ antagonist can act to prevent the inflammatory condition of gout.

Radiation-induced gastrointestinal inflammation is another inflammatory disorder believed to be mediated by LTB₄. Radiation works by damaging cancer cells, but unfortunately can damage non-diseased tissue as well, causing a typical inflammatory reaction in response. Therapeutic radiation is thus generally applied to a defined area of the subject's body which contains abnormal proliferative tissue in order to maximize the dose absorbed by the abnormal tissue and minimize the dose absorbed by the nearby normal tissue. However, it is difficult (if not impossible) to selectively administer therapeutic ionizing radiation to the abnormal tissue. Thus, normal tissue proximate to the abnormal tissue is also exposed to potentially damaging doses of ionizing radiation throughout the course of treatment. Moreover, some treatments that require exposure of the subject's entire body to the radiation, in a procedure called “total body irradiation”, or “TBI.” The efficacy of radiotherapeutic techniques in destroying abnormal proliferative cells is therefore necessarily balanced by the associated cytotoxic effects on nearby normal cells.

After or during a course of radiotherapy, LTB₄-mediated inflammatory processes may be triggered, causing damage to the bowel, and leading to sloughing of the cells of the inner lining of the GI tract. Radiation-induced gastrointestinal inflammation can present another circumstance wherein it is known beforehand that an individual will or is likely to develop an inflammatory disorder. In the instance of patients undergoing radiotherapy, the inflammation, damage and sloughing of the gastrointestinal tract is a predictable side effect of the radiotherapy.

New antiinflammatory agents are needed which are useful in the treatment of inflammatory disorders such as IBD, rheumatoid arthritis, gout, psoriasis and radiation-induced gastrointestinal inflammation. In particular, agents are needed that are appropriate for chronic long-term use in treatment. In addition, agents are needed that are useful in the prevention of LTB₄-mediated inflammatory disorders that occur secondary to observable events such as ionizing radiation therapy.

Thromboxane A₂

Thromboxane A₂ (TXA₂), like LTB₄, is a product of the arachidonic acid metabolic pathway. TXA₂ induces a variety of differential cellular responses including platelet aggregation, contraction of vascular and bronchial smooth muscle cells (SMC), potentiation of hypertrophic and mitogenic responses in vascular SMC and endothelial cells.

TXA₂ is considered to be an important mediator of asthma because it can induce contraction of airway smooth muscle, and because it has been implicated in airway hyperresponsiveness in animal models wherein increased airway reactivity was induced by allergens, platelet-activating factor (PAF), LTC₄, LTD₄, LTB₄, bradykinin, endothelin, endotoxin and ozone. (See J. Dogne et al., Expert Opin. Investig. Drugs (2002), 11(2), and references cited therein, the entire disclosures of which are incorporated herein by reference.)

TXA₂ has also been implicated in the pathophysiology of radicular pain induced by hemeated nucleus pulposis. A study in a rat model examined the role of TXA₂ (and LTB₄) in the hyperalgesia induced by application of nucleus pulposus to the lumbar nerve root in the rat. A TXA₂ synthetase inhibitor, injected into the epidural space, decreased mechanical hyperalgesia at both three and seven days after epidural injection. There were no significant differences in sensitivity to noxious thermal stimuli following application of the nucleus pulposus or an epidural injection. Epidural injection of TXA₂ synthetase inhibitor may attenuate the painful radiculopathy due to lumbar disc herniation.

TXA₂ has further been implicated as an in vivo mediator of fibroblast growth factor (FGF)-stimulated angiogenesis. See, T. Daniel et al., Cancer Research, 59, 4574-4577, Sep. 15, 1999, the entire disclosure of which is incorporated herein by reference. Thromboxane synthase inhibitors have further been shown to inhibit metastasis of lung carcinoma in a mouse model, thus demonstrating the involvement of TXA₂ in angiogenesis and tumor metastasis. See, D. Nie et al., Biochem. Biophys. Res. Commun., 2000, 267(1), p. 245-251, the entire disclosure of which is incorporated herein by reference.

TXA₂ is also believed to possess anticoagulant activity. See Schenk et al., “Antiplatelet and anticoagulant effects of “HN-11 500,” a selective thromboxane receptor antagonist,” Thromb. Res. 2001 Jul. 15;103(2):79-91.

Anticoagulant has potential therapeutic value in chronic inflammation according to a model associating chronic inflammatory disorders with a coagulation protein defect is termed immune system activation of coagulation (ISAC). The model proposes that a majority of individuals diagnosed with certain chronic inflammatory illnesses may, based on clinical criteria, be potentially defined as or involve AntiPhospholipid Antibody Syndrome (APS)—with the endothelial cell (EC) as the disease target. These patients have a hypercoagulable, state demonstrated by increased markers of coagulation activation and increased blood viscosity due to the generation of Soluble Fibrin Monomer (SFM). The CFS/FM process and related processes may be triggered by a variety of pathogens (CMV, HHV6, Mycoplasma, Chlamydia pneumonia, etc.), or some vaccines, resulting in pathogen-mediated immune activation that induces antibodies which cross react with EC protective proteins B₂GPI & Annexin V. These antibodies dislodge the protective proteins from EC surfaces, exposing PhosphatidylSerine (PS) on the EC surfaces in capillary beds.

Pathogens induce inflammatory responses which include cytokine modulation of EC to down regulate the antithrombotic environment (ThromboModulin, tPA) in favor of prothrombotic expression of Tissue Factor (TF). TF and PS exposure allows binding of the coagulation tenase and prothombinase complexes to EC surfaces. This results in thrombin generation leading to SFM formation. SFM dimerizes easily, increasing blood viscosity and precipitating out on EC surfaces as fibrin(oid) deposition, creating local ischemia and pathology, blocking nutrient and oxygen delivery in the microcirculation. A blood clot does not form because there is not enough of a thrombin burst to activate Factor XIII to cross link the fibrin into a clot.

A hereditary defect in a coagulation regulatory protein; such as protein C, protein S, Factor V^{L}, prothrombin gene mutation, Heparin Cofactor II, tPA, PAI-₁, Lp(a), or elevated Factor II, X, XII, or homacysteine is predispositional in greater than 75% of patients. Because this hypercoagulability does not result in an immediate thrombosis (100% occlusion), but rather in fibrin deposition (50-95%), it has been suggested that an appropriate name for this antiphospholipid antibody process would be Immune System Activation of Coagulation (ISAC) syndrome.

The ISAC model provides an explanation for the therapeutic benefits reported with low dose anticoagulant therapy (heparin or warfarin) in some of these patients. Diagnoses with published associations include: Chronic Fatigue. Syndrome/Fibromyalgia (CFS/FM), Infertility (Recurrent Fetal Loss and Fetal Wastage Syndromes), Osteonecrosis of the Jaw, Multiple Sclerosis (MS), Depression and Autism. Diagnoses under investigation include: Crohn's Disease and Inflammatory Bowel Disease (IBD), Late Lyme Disease, Sjogren's Syndrome (SS), Transient Ischemic Attack (TIA), Attention Deficit Disorder (ADD) and Parkinson's Disease. See Berg et al., “Chronic Fatigue Syndrome &/or Fibromyalgia as a variation of antiphospholipid antibody syndrome (APS): An explanatory model and approach to laboratory diagnosis,” Blood Coagulation and Fibrinolysis, 1999, 10:435-438.

New TXA₂ agents are needed which may be useful in the treatment of TXA₂-mediated disorders such as asthma, pain, tumors in which angiogenesis associated with the tumor is mediated by TXA₂, and in chronic inflammatory illnesses such as, for example Chronic Fatigue Syndrome/Fibromyalgia, IBD, Crohn's Disease, late Lyme disease and IBD.

### SUMMARY OF THE INVENTION

In one embodiment of the invention, there is provided a pharmaceutical composition comprising a pharmaceutically acceptable carrier and the compound 1-(3-hydroxy-4-methoxyphenyl)-4-methyl-5-ethyl-7,8-dimethoxy-5H-2,3-benzodiazepine, or a pharmaceutically acceptable salt thereof.

The compound has the formula:

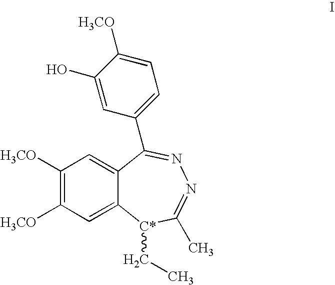

wherein C* is a chiral carbon and the bond designated by [symbol] indicates that the absolute conformation about C* may be either (R) or (S).

In another embodiment of the invention, a method of treating an inflammatory disorder mediated by LTB₄ is provided comprising administering to an individual in need of such treatment an effective amount of 1-(3-hydroxy-4-methoxyphenyl)-4-methyl-5-ethyl-7,8-dimethoxy-5H-2,3-benzodiazepine, or a pharmaceutically acceptable salt thereof.

In another embodiment of the invention, a method of preventing or delaying the onset of an inflammatory disorder mediated by LTB₄ is provided comprising administering to an individual who is at risk of developing such an inflammatory disorder, an effective amount of 1-(3-hydroxy-4-methoxyphenyl)-4-methyl-5-ethyl-7,8-dimethoxy-5H-2,3-benzodiazepine, or a pharmaceutically acceptable salt thereof.

In another embodiment of the invention, a method of treating a TXA₂-mediated disorder is provided, comprising administering to an individual in need of such treatment an effective amount of 1-(3-hydroxy-4-methoxyphenyl)-4-methyl-5-ethyl-7,8-dimethoxy-5H-2,3-benzodiazepine, or a pharmaceutically acceptable salt thereof.

In another embodiment of the invention, a method of preventing or delaying the onset of an inflammatory disorder mediated by TXA₂-in an individual who is at risk of developing an inflammatory disease state is provided, comprising administering an effective amount of 1-(3-hydroxy-4-methoxyphenyl)-4-methyl-5-ethyl-7,8-dimethoxy-5H-2,3-benzodiazepine, or a pharmaceutically acceptable salt thereof.

The invention also relates to the use in medicine of 1-(3-hydroxy-4-methoxyphenyl)-4-methyl-5-ethyl-7,8-dimethoxy-5H-2,3-benzodiazepine, the (R)- or (S)-enantiomers thereof, or pharmaceutically acceptable salts thereof.

According to another aspect of the invention, the aforesaid compounds are used in the preparation of medicaments for (i) treating an LTB₄-mediated inflammatory disorder, or for preventing or delaying the onset of such a disorder; (ii) treating a TXA₂-mediated disorder; and (iii) preventing or delaying the onset of an inflammatory disorder mediated by TXA₂.

In the compositions and methods discussed herein, the compound may comprise racemic-1-(3-hydroxy-4-methoxyphenyl)-4-methyl-5-ethyl-7,8-dimethoxy-5H-2,3-benzodiazepine, or the substantially isolated (R)- or (S)-enantiomer. Preferably, the administered compound is in the form of a single enantiomer of 1-(3-hydroxy-4-methoxyphenyl)-4-methyl-5-ethyl-7,8-dimethoxy-5H-2,3-benzodiazepine, which comprises 80% or more by weight of the total weight of the compound.

More preferably, the amount of a single enantiomer of 1-(3-hydroxy-4-methoxyphenyl)-4-methyl-5-ethyl-7,8-dimethoxy-5H-2,3-benzodiazepine is 85% or more by weight of the total weight of the compound, most preferably 90% or more by weight. In other embodiments, the enantiomeric purity is 95% or more, or even 99% or more by weight. According to one particular embodiment, the compound comprises 90% or more by weight of the (R)-enantiomer.

### Definitions

The terms “inflammation” and “inflammatory response” refer to a defense reaction of living tissue to injury. The response serves to contain and to repair the injury.

An “inflammatory disorder mediated by LTB₄” or a “LTB₄-mediated disorder”, means to a disorder resulting from an inflammatory response wherein LTB₄ mediation is implicated as a factor in the etiology or progression of the disorder by observation of LTB₄ presence at the site of the inflammation or by other evidence that LTB₄ is involved in the etiology or progression of the inflammatory aspect of the disorder.

The term “TXA₂-mediated disorder” means a disorder wherein TXA₂ mediation is implicated as a factor in the etiology or progression of the disorder or in the mechanisms whereby the disorder negatively affects the organism suffering therefrom.

The term “angiogenesis” means the process of vascularization of a tissue involving the development of new capillary blood vessels. Vascularization of tumors is usually a prelude to more rapid growth and often to metastasis.

The term “asthma” refers to a chronic respiratory disease, often arising from allergies, that is characterized by sudden recurring attacks of labored breathing, chest constriction, and coughing, due to due to a spasmodic contraction of the bronchi.

The phrase “optically active” refers to a property whereby a material rotates the plane of plane-polarized light. A compound that is optically active is nonsuperimposable on its mirror image. The property of nonsuperimposablity of an object on its mirror image is called chirality.

The property of “chirality” in a molecule may arise from any structural feature that makes the molecule nonsuperimposable on its mirror image. The most common structural feature producing chirality is an asymmetric carbon atom, i.e., a carbon atom having four nonequivalent groups attached thereto.

The term “enantiomer” refers to each of the two nonsuperimposable isomers of a pure compound that is optically active. Single enantiomers are designated according to the Cahn-Ingold-Prelog system, a set of priority rules that rank the four groups attached to an asymmetric carbon. See March, Advanced Organic Chemistry, 4^{th} Ed., (1992), p. 109. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example below, the Cahn-Ingold-Prelog ranking sequence id A>B>C>D. The lowest ranking atom, D is oriented away from the viewer.

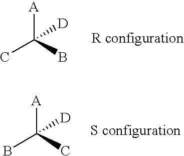

The term “racemate” or the phrase “racemic mixture” refers to a 50-50 mixture of two enantiomers such that the mixture does not rotate plane-polarized light.).

The term “substantially isolated”, or “substantially free” of the other enantiomer” or the term “resolved” when used to refer to an optically active compound of formula I, means the (R)- and (S)-enantiomers of the compound have been separated such that the composition is 80% or more by weight a single enantiomer.

Thus, by “(R)-1-(3-hydroxy-4-methoxyphenyl)-4-methyl-5-ethyl-7,8-dimethoxy-5H-2,3-benzodiazepine substantially free of the (S)-enantiomer” is meant 1-(3-hydroxy-4-methoxyphenyl)-4-methyl-5-ethyl-7,8-dimethoxy-5H-2,3-benzodiazepine that comprises 80% or more by weight of the (R)-enantiomer and likewise contains 20% or less of the (S)-enantiomer as a contaminant, by weight. Likewise, By “(S)-1-(3-hydroxy-4-methoxyphenyl)-4-methyl-5-ethyl-7,8-dimethoxy-5H-2,3-benzodiazepine substantially free of the (R)-enantiomer” is meant 1-(3-hydroxy-4-methoxyphenyl)-4-methyl-5-ethyl-7,8-dimethoxy-5H-2,3-benzodiazepine that comprises 80% or more by weight of the (S)-enantiomer and likewise contains 20% or less of the (R)-enantiomer as a contaminant, by weight.

The term “effective amount” when used to describe the amount of drug administered to a patient suffering from a LTB₄-mediated inflammatory disorder, refers to the amount of a compound that inhibits the inflammatory process, resulting in a therapeutically useful and selective reduction in the symptoms of inflammation when administered to a patient suffering from a disorder which manifests chronic or acute inflammation associated with physiologically relevant concentrations of LTB₄.

An “effective amount” of the compound when used to describe the amount of drug administered for the prevention of an LTB₄ mediated inflammatory disorder is an amount which prevents or delays the onset of symptoms of an inflammatory disorder in an individual during a time interval coinciding with an increased risk of LTB₄-mediated inflammatory disorder.

The term “effective amount” when used to describe therapy to a patient suffering from TXA₂-mediated pain, refers to the amount of 1-(3,4-dimethoxyphenyl)-4-methyl-5-ethyl-7-methoxy-8-hydroxy-5H-2,3-benzodiazepine that inhibits the process whereby pain is generated, thus resulting in a therapeutically useful and selective reduction in the pain sensation, when administered to a patient suffering from a disorder which manifests chronic or acute pain associated with physiologically relevant concentrations of TXA₂.

The term “effective amount” when used to describe therapy to a patient suffering from TXA₂-mediated angiogenesis in a tumor, refers to the amount of 1-(3,4-dimethoxyphenyl)-4-methyl-5-ethyl-7-methoxy-8-hydroxy-5H-2,3-benzodiazepine that inhibits the process whereby new blood vessels are generated that are associated with the developing tumor, thus resulting in a therapeutically useful and selective reduction rate of tumor development, when administered to a patient suffering from a tumor whose development is associated with physiologically relevant concentrations of TXA₂.

The term “effective amount” when used to describe therapy to a patient suffering from TXA₂-mediated asthma, refers to the amount of 1-(3,4-dimethoxyphenyl)-4-methyl-5-ethyl-7-methoxy-8-hydroxy-5H-2,3-benzodiazepine that ameliorates the symptoms of asthma, when administered to a patient suffering therefrom.

The term “effective amount” when used to describe the amount of drug administered to a patient who has suffered a stroke or other cerebral ischemic condition, or who is at elevated risk of suffering a stroke or other cerebral ischemic condition, refers to the amount of a compound that results in a therapeutically useful reduction or elimination of the neuronal cell death associated with such a disorder.

The term “effective amount” when used to describe the amount of drug administered to a patient suffering from epilepsy, refers to the amount of a compound that results in a therapeutically useful decrease in the frequency, the severity or both of the seizures associated with such a disorder.

The term “epilepsy” refers to any of various neurological disorders characterized by recurring attacks of motor, sensory, or psychic malfunction with or without loss of consciousness and with or without convulsive seizures.

The term “effective amount” when used to describe the amount of drug administered to a patient suffering from congestive heart failure, refers to the amount of a compound that results in a therapeutically useful decrease in the symptoms of heart failure, i.e., the shortness of breath, edema, fatigue associated with the failing heart.

The term “effective amount” when used to describe therapy to a patient suffering from myelosuppression associated with cytotoxic chemotherapy, e.g., cancer chemotherapy or ionizing radiation therapy, refers to the amount of 1-(3,4-dimethoxyphenyl)-4-methyl-5-ethyl-7-methoxy-8-hydroxy-5H-2,3-benzodiazepine that increases blood cell production, particularly granulocyte production, thereby resulting in a therapeutically useful and selective reduction of the myelosuppression in the individual undergoing the chemotherapy or ionizing radiation therapy.

The term “effective amount” when used to describe therapy to a patient at elevated risk of developing myelosuppression due to a present or imminent administration of cytotoxic chemotherapy, e.g., cancer chemotherapy, or ionizing radiation therapy, in order to prevent the secondary myelosuppression associated therewith, refers to the amount of 1-(3,4-dimethoxyphenyl)-4-methyl-5-ethyl-7-methoxy-8-hydroxy-5H-2,3-benzodiazepine that prevents, reduces or delays the reduction in blood cell production, particularly granulocyte production generally associated with such therapies.

The term “individual” or “subject” includes human beings and non-human animals. With respect to the disclosed methods of treating LTB₄-mediated inflammatory disorders, these terms refer, unless the context indicates otherwise, to an organism that is afflicted with such an inflammatory disorder.

With respect to disclosed methods of “preventing” or “delaying the onset” of LTB₄-mediated inflammatory disorders, these terms refer, unless the context indicates otherwise, to an organism that is likely to be afflicted with such an inflammatory disorder. The selection of an individual likely to incur such an inflammatory disorder may take into account the presence of inflammatory conditions that historically are known to have a high incidence of recurrence, such as, for example, IBD. The likelihood of incurring such an inflammatory disorder may also be due to tissue insult which is known beforehand, such as a surgical procedure. The future inflammatory disorder may also result from a secondary effect of an initial tissue insult. An example of this is inflammation due to gout caused by elevated uric acid levels that occur secondary to lysis of a tumor mass following administration of cytotoxic chemotherapy or therapeutic radiation treatment.

### DETAILED DESCRIPTION OF THE INVENTION

According to the present invention, 1-(3-hydroxy-4-methoxyphenyl)-4-methyl-5-ethyl-7,8-dimethoxy-5H-2,3-benzodiazepine, and pharmaceutically acceptable salts thereof, interact with the LTB₄ receptor and are useful in methods of treatment or prevention of inflammatory disorders mediated by LTB₄.

Such inflammatory disorders include, but are not limited to, Inflammatory Bowel Disease, including Crohn's Disease and ulcerative colitis; psoriasis; gout, rheumatoid arthritis and radiation-induced gastrointestinal inflammation.

In addition, 1-(3-hydroxy-4-methoxyphenyl)-4-methyl-5-ethyl-7,8-dimethoxy-5H-2,3-benzodiazepine, and pharmaceutically acceptable salts thereof, interact with the TXA₂ receptor and are thus useful in methods of treatment of disease processes mediated by TXA₂ including, but not limited to pain, asthma and angiogenesis associated with tumor development, and immune system activation of coagulation, and chronic inflammatory disorders.

Chronic inflammatory disorders believed to be treatable or preventable by administration of an effective amount of 1-(3-hydroxy-4-methoxyphenyl)-4-methyl-5-ethyl-7,8-dimethoxy-5H-2,3-benzodiazepine include, for example, chronic fatigue syndrome/fibromyalgia, infertility, osteonecrosis of the jaw, multiple sclerosis, depression, autism, Crohn's Disease, Inflammatory Bowel Disease, late Lyme Disease, Sjogren's Syndrome, transient ischemic attack, attention deficit disorder and Parkinson's Disease.

Preparation of 1-(3-Hydroxy-4-methoxyphenyl)-4-methyl-5-ethyl-7,8-dimethoxy-5H-2,3-benzodiazepine

The 1-(3-hydroxy-4-methoxyphenyl)-4-methyl-5-ethyl-7,8-dimethoxy-5H-2,3-benzodiazepine useful in the present invention may be prepared by one of several methods. These methods generally follow the synthetic strategies and procedures used in the synthesis of 2,3-benzodiazepines such as tofisopam and tofisopam analogs. See U.S. Pat. Nos. 3,736,315 and 4,423,044 (tofisopam syntheses) and Horvath et al., Progress in Neurobiology 60(2000) p.309-342 and references cited therein (preparation of tofisopam and analogs thereof), the entire disclosures of which are incorporated herein by reference. See also Kórósi et al., U.S. Pat. No. 4,322,346, the entire disclosure of which is incorporated herein by reference, disclosing three variations of the reaction protocol for preparing a substituted 2,3-benzodiazepine from the precursor benzopyrilium salt. A similar synthetic sequence for preparation of 2,3-benzodiazepinesis disclosed in U.S. Pat. No. 3,736,315, the entire disclosure of which is incorporated herein by reference, the entire disclosure of which is incorporated herein by reference. Alternative methods for preparation of the benzopyrilium intermediate start with an aryl acetonide or indanone starting material. See Kunnetsov, E. V., and Dorofeenko, G. N., Zh. Org Khim., 6, 578-581. and M. Vajda, Acta Chem. Acad. Sci. Hung, 40, p.295-307, 1964, respectively, the entire disclosures of which are incorporated herein by reference.

In the synthesis methods, the product of the chemical synthesis is racemic 1-(3-hydroxy-4-methoxyphenyl)-4-methyl-5-ethyl-7,8-dimethoxy-5H-2,3-benzodiazepine. This racemic mixture is optionally subsequently separated using known methods of resolution to produce (R)-1-(3-hydroxy-4-methoxyphenyl)-4-methyl-5-ethyl-7,8-dimethoxy-5H-2,3-benzodiazepine substantially free of the corresponding (S)-enantiomer, and (S)-1-(3-hydroxy-4-methoxyphenyl)-4-methyl-5-ethyl-7,8-dimethoxy-5H-2,3-benzodiazepine substantially free of the corresponding (R)-enantiomer.

Resolution of 1-(3-hydroxy-4-methoxyphenyl)-4-methyl-5-ethyl-7,8-dimethoxy-5H-2,3-benzodiazepine.

Use of the synthetic procedures referenced above will effect preparation of racemic 1-(3-hydroxy-4-methoxyphenyl)-4-methyl-5-ethyl-7,8-dimethoxy-5H-2,3-benzodiazepine. The racemate must be resolved in order to isolate the individual (R)- and (S)-enantiomers of 1-(3-hydroxy-4-methoxyphenyl)-4-methyl-5-ethyl-7,8-dimethoxy-5H-2,3-benzodiazepine. Enantiomeric resolution may be achieved by converting racemic 1-(3-hydroxy-4-methoxyphenyl)-4-methyl-5-ethyl-7,8-dimethoxy-5H-2,3-benzodiazepine to a pair of diastereomers by either covalently bonding to an optically active moiety, or by salt formation with an optically active base or acid. Either of these two methods provides a molecule with a second chiral center, thus generating a pair of diastereomers. This diastereomeric pair is then separated by conventional methods such as for example, crystallization or chromatography.

Racemic 1-(3-hydroxy-4-methoxyphenyl)-4-methyl-5-ethyl-7,8-dimethoxy-5H-2,3-benzodiazepine may be converted to the (S)-dibenzoyltartaric acid salt, which is a diastereomeric mixture of SS and RS configurations. The pair of diastereomers (R,S) and (S,S) possess different properties, e.g., differential solubilities, that allow for the use of conventional separation methods. Fractional crystallization of diastereomeric salts from a suitable solvent is one such separation method. This resolution has been successfully applied to the resolution of racemic tofisopam. See Hungarian Patent 178516 and also Toth et al., J. Heterocyclic Chem., 20:09-713 (1983), the entire disclosures of which are incorporated herein by reference.

Alternatively, racemic-1-(3-hydroxy-4-methoxyphenyl)-4-methyl-5-ethyl-7,8-dimethoxy-5H-2,3-benzodiazepine may be derivatized via, for example, acylation of the 3′-hydroxy moiety with a chiral acylating reagent such as, for example, (S)-mandelic acid. The resulting ester, has a second chiral center, and thus exists as a diastereomeric pair separable using conventional methods such as crystallization or chromatography. Following the separation, the chiral moiety with which 1-(3-hydroxy-4-methoxyphenyl)-4-methyl-5-ethyl-7,8-dimethoxy-5H-2,3-benzodiazepine was derivatized, may be removed.

Racemic 1-(3-hydroxy-4-methoxyphenyl)-4-methyl-5-ethyl-7,8-dimethoxy-5H-2,3-benzodiazepine may be separated without diastereomer formation by differential absorption on a chiral stationary phase of a chromatography column, particularly a preparative HPLC column. Chiral HPLC columns are commercially available with a variety of packing materials to suit a broad range of separation applications. Exemplary stationary phases suitable for resolving the racemic 2,3-benzodiazepines include:

(i) macrocyclic glycopeptides, such as silica-bonded vancomycin which contains 18 chiral centers surrounding three pockets or cavities;

(ii) chiral α₁-acid glycoprotein;

(iii) human serum albumin; and

(iv) cellobiohydrolase (CBH).

Chiral α₁-acid glycoprotein is a highly stable protein immobilized onto spherical silica particles that tolerates high concentrations of organic solvents, high and low pH, and high temperatures. Human serum albumin, though especially suited for the resolution of weak and strong acids, zwitterionic and nonprotolytic compounds, has been used to resolve basic compounds. CBH is a very stable enzyme which has been immobilized onto spherical silica particles and is preferentially used for the separation of enantiomers of basic drugs from many compound classes.

The resolution of tofisopam by chiral chromatography using macrocyclic glycopeptide as a stationary phase on a Chirobiotic V™ column (ASTEAC, Whippany, N.J.) is disclosed in U.S. Pat. No. 6,080,736. Fitos et al. (J. Chromatogr., 709 265 (1995)), discloses another method for resolving racemic tofisopam by chiral chromatography using a chiral α₁-acid glycoprotein as a stationary phase on a CHIRAL-AGP™ column (ChromTech, Cheshire, UK). The latter method separates the (R)- and (S)-enantiomers and also resolves the two conformers (discussed below) of each enantiomer. These chromatographic methods, may be used generally to separate racemic 2,3-benzodiazepines into individual (R)- and (S)-enantiomers. The Chirobiotic V™ column is available in a semi-preparative size as employed for the above separation 500 mm×10 mm). The stationary phase of the Chirobiotic V™ column is commercially available in bulk for packing of preparative chromatography columns with larger sample capacity.

(R)- and (S)-enantiomers of 2,3-benzodiazepines may also exist in two stable conformations that may be assumed by the benzodiazepine ring, as generally depicted below:

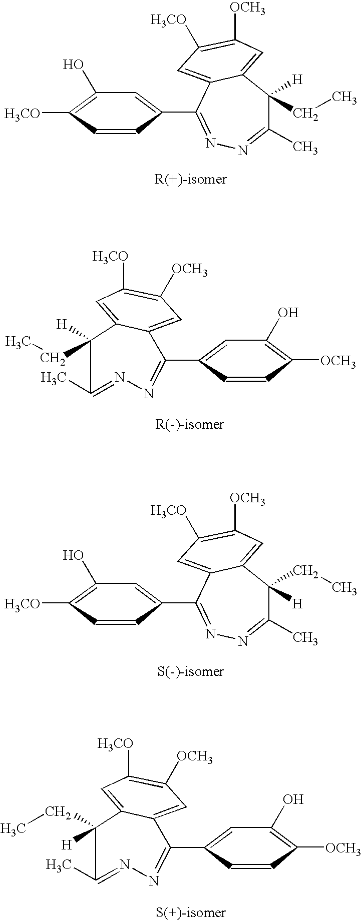

The present invention includes compositions and methods as described herein that use any and all observable conformations of 1-(3-hydroxy-4-methoxyphenyl)-4-methyl-5-ethyl-7,8-dimethoxy-5H-2,3-benzodiazepine.

The compound used in the compositions and methods of the present invention may take the form of a pharmaceutically-acceptable salt. The term “salts”, embraces salts commonly used to form alkali metal salts and to form addition salts of free acids or free bases. The term “pharmaceutically-acceptable salt” refers to salts that possess toxicity profiles within a range so as to have utility in pharmaceutical applications. Pharmaceutically unacceptable salts may nonetheless possess properties such as high crystallinity, which have utility in the practice of the present invention, such as for example utility in a synthetic process or in the process of resolving enantiomers from a racemic mixture. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, salicyclic, galactaric and galacturonic acid.

Suitable pharmaceutically acceptable base addition salts of racemic-1-(3-hydroxy-4-methoxyphenyl)-4-methyl-5-ethyl-7,8-dimethoxy-5H-2,3-benzodiazepine, or the (R)- or (S)-enantiomer thereof, include for example, metallic salts made from calcium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from 1-(3-hydroxy-4-methoxyphenyl)-4-methyl-5-ethyl-7,8-dimethoxy-5H-2,3-benzodiazepine by reacting, for example, the appropriate acid or base with 1-(3-hydroxy-4-methoxyphenyl)-4-methyl-5-ethyl-7,8-dimethoxy-5H-2,3-benzodiazepine.

The compound useful in the compositions and methods of the invention may be administered to individuals (mammals, including animals and humans) afflicted with LTB₄-mediated inflammatory disorders or disorders mediated by TXA₂. The latter include, but not limited to, pain, asthma and tumor development which involves angiogenesis mediated by TXA₂.

For treating or preventing inflammatory disorders mediated by LTB₄, or for treating disorders mediated by TXA₂, the specific dose of racemic-1-(3-hydroxy-4-methoxyphenyl)-4-methyl-5-ethyl-7,8-dimethoxy-5H-2,3-benzodiazepine, or an enantiomer thereof, to obtain therapeutic benefit, is determined by the particular circumstances of the individual patient including, the size, weight, age and sex of the patient. Also determinative is the nature and stage of the disease and the route of administration. Generally, a daily dosage of from about 100 to 1500 mg/kg/day may be utilized. Preferably, a daily dosage of from about 100 to 1000 mg/kg/day may be utilized. More preferably, a daily dosage of from about 100 to 500 mg/kg/day may be utilized. Higher or lower doses are also contemplated.

For prophylactic administration, the compound should be administered far enough in advance of a known event that increases the chance of an inflammatory disorder mediated by LTB₄ such that the compound is able to reach the site of action in sufficient concentration to exert an effect modulating LTB₄ activity. The pharmacokinetics of specific compounds may be determined by means known in the art and tissue levels of a compound in a particular individual may be determined by conventional analyses.

Likewise, for prophylaxis involving a disorder mediated by TXA₂, the timing of compound administration should take into account factors relating to a recurrent condition such as asthma, and to events reasonably expected to trigger pain, symptoms, such as post-operative pain or pain caused by a progressive disorder.

The compositions of the present invention comprise a pharmaceutically acceptable carrier and: (i) racemic-1-(3-hydroxy-4-methoxyphenyl)-4-methyl-5-ethyl-7,8-dimethoxy-5H-2,3-benzodiazepine, (ii) (R)-1-(3-hydroxy-4-methoxyphenyl)-4-methyl-5-ethyl-7,8-dimethoxy-5H-2,3-benzodiazepine substantially free of the corresponding (S)-enantiomer, (iii) (S)-1-(3-hydroxy-4-methoxyphenyl)-4-methyl-5-ethyl-7,8-dimethoxy-5H-2,3-benzodiazepine substantially free of the corresponding (R)-enantiomer, or a pharmaceutically acceptable salt of (i), (ii) or (iii). The active ingredient in such formulations may comprise from 0.1 to 99.99 weight percent. By “pharmaceutically acceptable carrier” is meant any carrier, diluent or excipient that is compatible with the other ingredients of the formulation and not deleterious to the recipient.

The compound may be administered for therapeutic effect by any route, for example enteral (e.g., oral, rectal, intranasal, etc.) and parenteral administration. Parenteral administration includes, for example, intravenous, intramuscular, intraarterial, intraperitoneal, intravaginal, intravesical (e.g., into the bladder), intradermal, topical or subcutaneous administration. Also contemplated within the scope of the invention is the instillation of drug in the body of the patient in a controlled formulation, with systemic or local release of the drug to occur at a later time. For antiinflammatory use, the drug may be localized in a depot for controlled release to the circulation, or controlled release to a local site of inflammation.

The pharmaceutically acceptable carrier is selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agent. Stabilizing agents, antioxidizing agents and preservatives may also be added. Suitable antioxidizing agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The compositions of the present invention may also be formulated so as to provide slow or controlled-release of the active ingredient therein. In general, a controlled-release preparation is a composition capable of releasing the active ingredient at the required rate to maintain constant pharmacological activity for a desirable period of time. Such dosage forms may provide a supply of a drug to the body during a predetermined period of time and thus maintain drug levels in the therapeutic range for longer periods of time than other non-controlled formulations.

For example, U.S. Pat. No. 5,674,533 discloses controlled-release compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 discloses a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 discloses controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. The patents cited above are incorporated herein by reference.

Biodegradable microparticles may be used in the controlled-release formulations of this invention. For example, U.S. Pat. No. 5,354,566 discloses a controlled-release powder that contains the active ingredient. U.S. Pat. No. 5,733,566 describes the use of polymeric microparticles that release antiparasitic compositions. These patents are incorporated herein by reference.

The controlled-release of the active ingredient may be stimulated by various inducers, for example pH, temperature, enzymes, water, or other physiological conditions or compounds. Various mechanisms of drug release exist. For example, in one embodiment, the controlled-release component can swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient (e.g., 1-(3-hydroxy-4-methoxyphenyl)-4-methyl-5-ethyl-7,8-dimethoxy-5H-2,3-benzodiazepine or a pharmaceutically-acceptable salt thereof) in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The practice of the invention is illustrated by the following non-limiting examples.

### EXAMPLES

### Example 1

### Synthesis of racemic-1-(3-hydroxy-4-methoxyphenyl)-4-methyl-5-ethyl-7,8-dimethoxy-5H-2,3-benzodiazepine

Racemic-1-(3-hydroxy-4-methoxyphenyl)-4-methyl-5-ethyl-7,8-dimethoxy-5H-2,3-benzodiazepine was synthesized according to the route of Scheme 3.

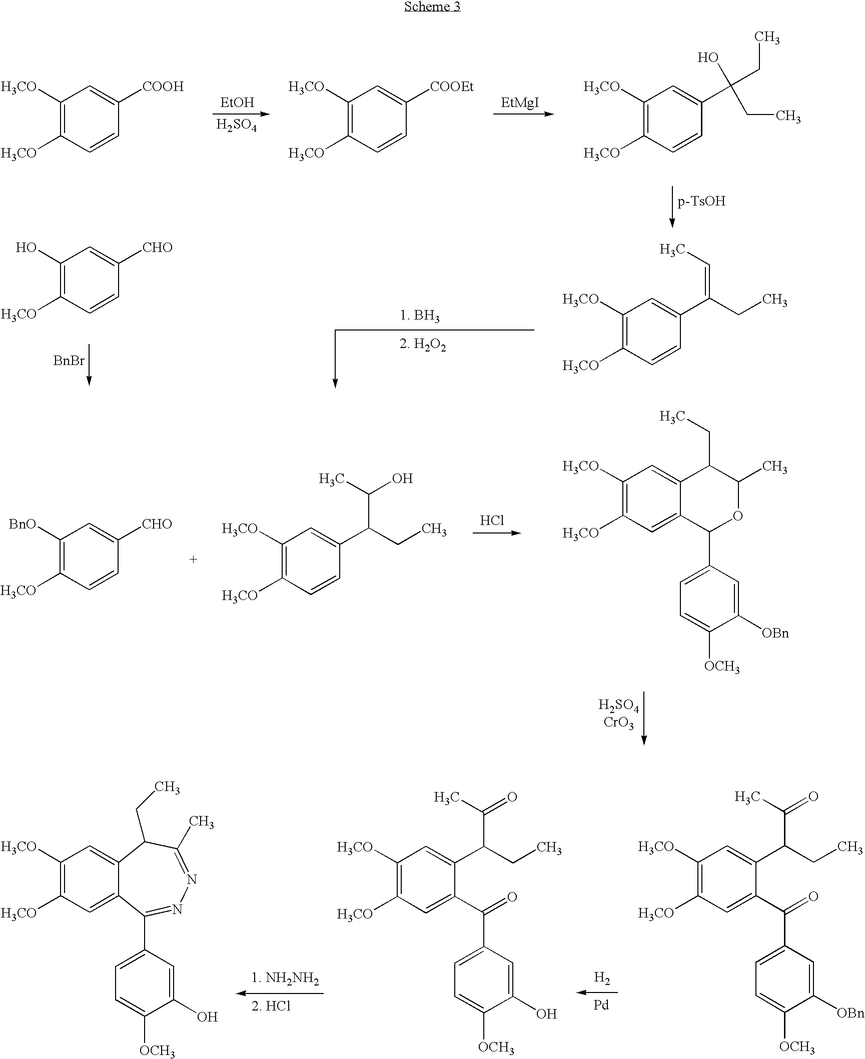

A. Esterification of 3,4-dimethoxybenzoic acid to Yield ethyl-3,4-dimethoxybenzoate ([3943-77-9]).

A solution of 200 g of 3,4-dimethoxybenzoic acid and 35 g of concentrated sulfuric acid in 600 mL of absolute ethanol was heated at reflux overnight. The mixture was concentrated and the residue poured into water. Methylene chloride was added and the solution washed successively with water, dilute sodium bicarbonate and water, then dried and concentrated. The residue was recrystallized from acetone/hexane.

B. Addition of Ethyl Magnesium Iodide to ethyl-3,4-dimethoxybenzoate acid to Yield 3-(3,4-dimethoxyphenyl)pentan-3-ol.

A solution of 4.8 mL of iodoethane in 20 mL of ether was added dropwise to a suspension of 1.5 g of magnesium turnings in 10 mL of ether. After 5 mL of the iodoethane solution had been added, a few grains of iodine were added and the mixture was heated to induce formation of the Grignard reagent. The remaining iodoethane solution was then added. After the Grignard formation was complete, a solution of 5 g of ethyl 3,4-dimethoxybenzoate in ether was added and the mixture was allowed to stir at room temperature overnight. The reaction was quenched by addition of saturated ammonium chloride. The mixture was extracted with ether. The combined ether extracts were dried and concentrated to an oily residue. Yield: 5 g.

C. Elimination of H₂O from 3-(3,4-dimethoxyphenyl)pentan-3-ol to Yield 4-((1 Z)-1-ethylprop-1-enyl)-1,2-dimethoxybenzene.

A solution of 5 g of crude 3-(3,4-dimethoxyphenyl)pentan-3-ol and 0.25 g of p-tolenesulfonic acid in 80 mL of benzene was heated at reflux for 1 hr with azeotropic removal of water. The mixture was then filtered through a pad of sodium bicarbonate and the filtrate concentrated. The residue was purified by distillation under reduced pressure. Yield: 2.9 g.

D. Addition of H₂O to 4-((1Z)-1-ethylprop-1-enyl)-1,2-dimethoxybenzene to Yield 3-(3,4-dimethoxyphenyl)pentan-2-ol.

To a solution of 26 g of 4-((1Z)-1-ethylprop-1-enyl)-1,2-dimethoxybenzene in tetrahydrofuran at 0° C. was added 189 mL of a 1.0M solution of borane-tetrahydrofuran complex in tetrahydrofuran. The mixture was stirred for 3 hr at 0° C., then 35.6 mL of 50% hydrogen peroxide was added, with simultaneous addition of 5M sodium hydroxide to maintain the mixture at pH 8. The mixture was extracted with ether. The combined ether extracts were dried and concentrated.

E. Benzylation of 3-hydroxy-4-methoxybenzaldehyde to Yield 4-methoxy-3-(phenylmethoxy)benzaldehyde ([6346-05-0]).

A solution of 100 g of 3-hydroxy-4-methoxybenzaldehyde and 135 g of benzyl bromide in 500 mL of acetone containing a suspension of 137 g of potassium carbonate was heated at reflux overnight. The mixture was filtered, the filtrate concentrated and the residue recrystallized from toluene/hexane. Yield: 65 g.

F. Reaction of 3-(3,4-dimethoxyphenyl)pentan-2-ol with 4-methoxy-3-(phenylmethoxy)benzaldehyde to Yield 4-(4-ethyl-6,7-dimethoxy-3-methyliso-chromanyl)-1-methoxy-2-(phenylmethoxy)benzene.

A solution of 14 g of 4-methoxy-3-(phenylmethoxy)benzaldehyde and 15 g of 3-(3,4-dimethoxyphenyl)pentan-2-ol in 0.3 L of dioxane was saturated with hydrogen chloride gas. The mixture was heated at reflux for 3 hr, saturated again with hydrogen chloride gas and allowed to stir at room temperature overnight. It was then poured into water, basified with dilute sodium hydroxide and extracted with methylene chloride. The combined methylene chloride extracts were dried and concentrated.

G. Ring-Opening of 4-(4-ethyl-6,7-dimethoxy-3-methyliso-chromanyl)-1-methoxy-2-(phenylmethoxy)benzene to Yield 3-(4,5-dimethoxy-2-{[4-methoxy-3-(phenylmethoxy)phenyl]carbonyl}phenyl)pentan-2-one.

To a solution of 30 g of crude 4-(4-ethyl-6,7-dimethoxy-3-methyliso-chromanyl)-1-methoxy-2-(phenylmethoxy)benzene in 450 mL of acetone at 5° C. was added a solution of 30 g of chromic oxide in 300 mL of 35% sulfuric acid. The mixture was stirred at room temperature for 2 hr, neutralized by adding cold 10% sodium hydroxide and concentrated to remove acetone. Then, water was added and the mixture was extracted with methylene chloride. The combined methylene chloride extracts were dried and concentrated. The residue was purified by column chromatography on silica gel. Yield: 10 g

H. Debenzylation of 3-(4,5-dimethoxy-2-{[4-methoxy-3-(phenylmethoxy)phenyl]carbonyl} phenyl)pentan-2-one to Yield 3-{2-[(3-hydroxy-4-methoxyphenyl)carbonyl]-4,5-dimethoxyphenyl} pentan-2-one.

A solution of 10 g of 3-(4,5-dimethoxy-2-{[4-methoxy-3-(phenylmethoxy)-phenyl]carbonyl}phenyl)pentan-2-one in methylene chloride containing a suspension of 0.9 g of 10% palladium on carbon was hydrogenated at 80 psi for 1 hr. The mixture was filtered through diatomaceous earth and the filtrate concentrated. Yield: 6.5 g

I. Annulation of 3-{2-[(3-hydroxy-4-methoxyphenyl)carbonyl]-4,5-dimethoxyphenyl}pentan-2-one by Reaction with Hydrazine to Yield 1-(3-hydroxy-4-methoxyphenyl)-4-methyl-5-ethyl-7,8-dimethoxy-5H-2,3-benzodiazepine.

A solution of 6.5 g of 3-{2-[(3-hydroxy-4-methoxyphenyl)carbonyl]-4,5-dimethoxyphenyl}pentan-2-one and 2.2 mL of hydrazine in 130 mL of ethanol was heated at reflux for 0.5 hr. After allowing the solution to cool to room temperature, it was saturated with HCl gas. The mixture was then concentrated to a volume of about 5 mL, basified with concentrated ammonium hydroxide, and extracted with methylene chloride. The combined methylene chloride extracts were dried and concentrated, and the residue recrystallized from ethyl acetate/hexane. Yield: 0.97 g.

The product 1-(3-hydroxy-4-methoxyphenyl)-4-methyl-5-ethyl-7,8-dimethoxy-5H-2,3-benzodiazepine was analyzed by HPLC, elemental analysis, GC/MS, proton NMR and differential scanning calorimetry (DSC). The data are as follows:

Purity: 99.29% by HPLC (% area). Column: Betasil Phenyl 4.6×150 mm. Mobile Phase: Acetonitrile::0.01M Phosphate Buffer (70::30). Flow Rate: 0.5 mL/min. Wavelength: 254 nm.

GC-MS; M/e=358; with the fragmentation pattern matching the proposed structure.

DSC: Temperature program 100° C. to 300° C. at 5° C./min, indicated molar purity=99.75% and melting point of 158.6° C.

Elemental analysis (calculated/analysis): % C—68.09/68.08; % H—6.61/6.57; N—7.53/7.35. Calculated values include 0.02 equivalents of ethyl acetate and 0.09 equivalents of residual water.

NMR (DCCl₃) (performed on GE QE 300): 1.08 ppm (t, 3H); 1.99 (s, 3H); 2.11 (m, 2H); 2.75 (m, 1H); 3.75 (s, 3H); 3.93 (s, 3H); 3.97 (s, 3H); 6.46 (bs, 1H); 6.72 (s, 1H); 6.86 (m, 2H); 7.18 (d, 1H); 7.48 (s, 1H).

### Example 2

### Resolution of 1-(3-hydroxy-4-methoxyphenyl)-4-methyl-5-ethyl-7,8-dimethoxy-5H-2,3-benzodiazepine

The enantiomers of racemic-1-(3-hydroxy-4-methoxyphenyl)-4-methyl-5-ethyl-7,8-dimethoxy-5H-2,3-benzodiazepine are resolved by chiral chromatography as follows.

Racemic-1-(3-hydroxy-4-methoxyphenyl)-4-methyl-5-ethyl-7,8-dimethoxy-5H-2,3-benzodiazepine is loaded onto a semipreparative (500 mm×10 mm) Chirobiotic V column (ASTEC, Whippany, N.J.). Elution of the enantiomeric mixture with methyl-tert-butyl ether/acetonitrile (90/10 V/V), at a flow rate of 40 mL/min, is monitored at 310 nm. Fraction size is 10-20 mL and fractions are subjected to analytical chromatography using the same solvent composition on an analytical (150×4.6 mm) Chirobiotic V column. The fractions containing each isolated enantiomer are processed by removing the elution solvent in vacuo.

### Example 3

### Inhibition of LTB₄ Binding

The ability of 1-(3-hydroxy-4-methoxyphenyl)-4-methyl-5-ethyl-7,8-dimethoxy-5H-2,3-benzodiazepine to inhibit [³H]LTB₄ binding to the LTB₄ receptor was determined as follows using the guinea pig spleen membrane assay of Cheng et al., J. Pharmacol. Exp. Ther., 236(1), 126-132, 1986, the entire disclosure of which is incorporated herein by reference.

Reactions were carried out in a phosphate buffer (pH 7.4) containing NaCl, MgCl₂, EDTA, and bacitracin. The reaction volume of 150 μL containing 1.0 mg/mL of the Guinea pig spleen membrane preparation and 1 nM [³H]LTB₄, with or without a candidate inhibitor, was incubated at 0-4° C. for 2 hours. Candidate inhibitors included the compounds listed in Table 1 and unlabeled LTB₄ as a control. The reaction was terminated by rapid vacuum filtration onto glass fiber filters. The filter was washed with cold buffer, dried and placed in a scintillation vial. Radioactivity trapped onto the filters was determined and compared to control values in order to ascertain any interactions of the test compound with the LTB₄ binding site.

As shown in Table 1, below, 1-(3-hydroxy-4-methoxyphenyl)-4-methyl-5-ethyl-7,8-dimethoxy-5H-2,3-benzodiazepine demonstrated a 64% inhibition of [³H]LTB₄ binding to the LTB₄ receptor at a concentration of 10 μM.

These binding results indicate that 1-(3-hydroxy-4-methoxyphenyl)-4-methyl-5-ethyl-7,8-dimethoxy-5H-2,3-benzodiazepine is useful in the treatment and prevention of disorders which are mediated by LTB₄.

TABLE 1
Summary of [³H]LTB₄ binding data
for 1-(3-hydroxy-4-methoxyphenyl)-4-methyl-5-ethyl-7,
8-dimethoxy-5H-2,3-benzodiazepine
compared with other 2,3-benzodiazepines

| | LTB₄ % inhib |
|---|---|
| Compound Name | @ 10 μm |
| 1-(3-hydroxy-4-methoxyphenyl)-4-methyl-5-ethyl-7,8- | 63.94 |
| dimethoxy-5H-2,3-benzodiazepine | |
| 1-(3,4-dimethoxyphenyl)-4-methyl-5-ethyl-7-methoxy-8- | 35.96 |
| hydroxy-5H-2,3-benzodiazepine | |
| 1-(3-methoxy-4-hydroxyphenyl)-4-methyl-5-ethyl-7,8- | 16.75 |
| dimethoxy-5H-2,3-benzodiazepine | |
| 1-(3,4-dimethoxyphenyl)-4-methyl-5-ethyl-7-hydroxy-8- | 20.35 |
| methoxy-5H-2,3-benzodiazepine | |
| 1-(3-methoxy-4-hydroxyphenyl)-4-methyl-5-ethyl-7- | inactive |
| hydroxy-8-methoxy-5H-2,3-benzodiazepine | |
| 1-(3-hydroxy-4-methoxyphenyl)-4-methyl-5-ethyl-7- | inactive |
| hydroxy-8-methoxy-5H-2,3-benzodiazepine | |

### Example 4

### TXA₂ Binding Assay

The ability of 1-(3-hydroxy-4-methoxyphenyl)-4-methyl-5-ethyl-7,8-dimethoxy-5H-2,3-benzodiazepine to inhibit the binding of [³H]SQ 29,548 (30-60 Ci/mmol) was determined via the human platelet-based assay of Hedberg et al., “Characterization of [³H]SQ 29,548 as a High Affinity Radioligand Binding to Thromboxane A₂—Receptors in Human Platelets.”, J. Pharmacol. Exp. Ther. 245: 786-792 (1988), with modifications. TXA₂ is a very unstable molecule, thus a surrogate ligand of known affinity for the TXA₂ receptor is required as a standard for determination of binding affinity of new potential TXA₂ ligands. [³H]SQ 29,548 is a ligand with known binding affinity for the TXA₂ receptor. [³H]SQ 29,548 has been employed as a TXA₂ ligand in several published studies, is accepted as a TXA₂ binding standard, and is thus useful as a standard in assessing the binding affinity of new compounds to the TXA₂ receptor. See also, Armstrong, R. A., Jones, R. L., et al. “Ligand Binding to Thromboxane Receptors on Human Platelets: Correlation with Biological Activity.” Brit. Jrnl. Pharmac. 79:953-964 (1983), the entire disclosures of which are incorporated herein by reference.

Reactions were carried out in a reaction mixture comprising 25 mM TRIS-HCl (pH 7.4) containing 138 mM NaCl, 5 mM KCl, 5 mM MgCl₂, 5.5 mM dextrose, and 2 mM EDTA at 25° C. for 60 minutes. Pinane-thromboxane (Kᵢ=149.0 nM) was employed as a competitor. The reaction was terminated by rapid vacuum filtration of the reaction mixture onto glass fiber filters. Radioactivity trapped onto the filters was determined and compared to control values in order to ascertain any interactions of test compound with the thromboxane A₂ binding site.

The compound 1-(3-hydroxy-4-methoxyphenyl)-4-methyl-5-ethyl-7,8-dimethoxy-5H-2,3-benzodiazepine demonstrated a 25.95% inhibition of [³H]SQ 29,548 binding to the TXA₂ receptor at a concentration of 10 μM.

These binding results indicate that 1-(3-hydroxy-4-methoxyphenyl)-4-methyl-5-ethyl-7,8-dimethoxy-5H-2,3-benzodiazepine is useful in the treatment and prevention of disorders which are mediated by TXA₂.

### Example 5

### Dextran Sulfate Sodium Induced Colitis: Mouse Model of Inflammatory Bowel Disease

In this model of colitis, an acute inflammation of the colon was produced by oral administration of dextran sulfate sodium (DSS) as a 5% solution in tap water. This colitis was characterized by histological events and an influx of neutrophils, macrophages and mediators of inflammation similar to those observed with human inflammatory bowel diseases. Several drugs known to be of useful for treating IBD, such as corticosteroids and 5-ASA, have been shown to have activity in this model. The following study was conducted in accordance with protocols of Okayasu et al., Gastroenterology, 98:694-702, 1990.

Sixty test animals (female, 6 week old Swiss Webster mice, 18-30 g) were divided into six groups, selected to eliminate any statistical differences in mean group weight. Each animal was dosed daily (PO) with either the test compound 1-(3-hydroxy-4-methoxyphenyl)-4-methyl-5-ethyl-7,8-dimethoxy-5H-2,3-benzodiazepine, a positive control (sulfasalazine), or a negative control (vehicle, comprising 0.5% carboxymethylcellulose (CMC) in distilled water), starting on Day 0. Dosing was by oral administration using a ball-tipped needle at a dose volume of 10 mL/kg.

Beginning on Day 1, acute colon inflammation was induced by the administration of DSS ad libitum in drinking water as a 5% solution in tap water (10 mL/mouse/day for 5-6 days). No other fluid source was available to the animals in the DSS arm of the study (groups 2-6). Filtered tap water was available ad libitum to another group (Group 1). After four days, signs of acute disease occurred with the loss of weight, diarrhea and bloody stools in the DSS treated animals. Histological changes included initial shortening of the crypts, then areas of separation of the crypts and the muscularis mucosae in the absence of destructive inflammatory filtrate. After five days, pathological changes became confluent with the appearance of erosions and early hyperplastic epithelium. Inflammation scores were high with neutrophils, lymphocytes, and plasma cells in the lamina propria but sparing the epithelium.

The test compound, the positive control (sulfasalazine), and the negative control standard (vehicle) were administered orally (PO). The test compound given during this period was evaluated for prophylactic activity and test compound given after the disease state was established was evaluated for therapeutic activity. Ten test animals were assigned to each of six dose groups listed in Table 2.

TABLE 2

| Group | Test substance (dose) | DSS or control |
|---|---|---|
| 1 | Vehicle IP daily | +tap water |
| 2 | Vehicle IP daily | +DSS 5% in tap water |
| 3 | Sulfasalazine (300 mg/kg IP daily) | +DSS 5% in tap water |
| 4 | 1-(3-hydroxy-4-methoxyphenyl)-4-methyl-5- | +DSS 5% in tap water |
| | ethyl-7,8-dimethoxy-5H-2,3-benzodiazepine (64 mg/kg | |
| | IP daily) | |
| 5 | 1-(3-hydroxy-4-methoxyphenyl)-4-methyl-5- | +DSS 5% in tap water |
| | ethyl-7,8-dimethoxy-5H-2,3-benzodiazepine (32 mg/kg | |
| | IP daily) | |
| 6 | 1-(3-hydroxy-4-methoxyphenyl)-4-methyl-5- | +DSS 5% in tap water |
| | ethyl-7,8-dimethoxy-5H-2,3-benzodiazepine (16 mg/kg | |
| | IP daily) | |

Test animals were weighed daily from Day 0 to Day 8, or until completion of the study. The total duration of the DSS arm of the study was varied depending on the time progress of colitis. The condition of the test animals and consistency of stools was noted.

At the conclusion of the study, test animals were euthanized (CO₂), a midline incision was made and a stool sample was obtained. The sample was placed on a slide and tested for occult blood (Quic-Cult, Laboratory Diagnostics Co., Morganville, N.J.). Occult blood was determined by placing two drops of the reagent onto the sample and observing any color change. Occult blood presence was graded using a scoring protocol assigning a score of 0 for no color; 1 for a very light blue color (+/−) forming in >30 seconds; 2 for a blue color developing in 30 seconds or more (+); 3 for a change in color occurring in less than 30 seconds (++); and 4 for gross blood observable on the slide. The colon was gently stretched and the length from the colon-cecal junction to the end of the distal rectum was measured to the nearest 0.1 cm. A Disease Activity Index (DAI) was determined. Table 3 lists scoring criteria for determination of the DAI.

TABLE 3

| Score | Weight loss (%) | Stool consistency | Blood in feces |
|---|---|---|---|
| 0 | 0 or gain | Normal | Negative |
| 1 | 1-4.9 | Soft | Hemoccult |
| | | | +/− |
| 2 | 5.0-9.9 | Mixed | Hemoccult + |
| | | (soft and diarrhea) | |
| 3 | 10-15 | Diarrhea | Hemoccult |
| | | | ++ |
| 4 | >15 | bloody diarrhea | gross blood |

The scores for each test animal were added and then divided by three to provide a DAI score for each animal. The data for the six groups is summarized in Tables 4, 5 and 6. Table 4 lists the DAI and the DAI without the weight loss parameter (DAIWT) for test animals in DSS-induced colitis study.

TABLE 4

| | | | Mean | Mean |
|---|---|---|---|---|
| Test substance | n | Group | DAI ± SEM | DAIWT ± SEM |
| Vehicle IP daily + water | 10 | 1 | 0.07 ± 0.04** | 0.10 ± 0.07** |
| Vehicle IP daily + DSS | 8 | 2 | 3.13 ± 0.27 | 3.13 ± 0.30 |
| Sulfasalazine (300 mg/kg IP daily) + DSS | 7 | 3 | 2.86 ± 0.18 | 2.50 ± 0.19 |
| 1-(3-hydroxy-4-methoxyphenyl)-4-methyl-5- | 7 | 4 | 2.06 ± 0.48 | 2.00 ± 0.45 |
| ethyl-7,8-dimethoxy-5H-2,3-benzodiazepine | | | | |
| (64 mg/kg IP daily) + DSS | | | | |
| 1-(3-hydroxy-4-methoxyphenyl)-4-methyl-5- | 8 | 5 | 2.67 ± 0.36 | 2.56 ± 0.27 |
| ethyl-7,8-dimethoxy-5H-2,3-benzodiazepine | | | | |
| (32 mg/kg IP daily) + DSS | | | | |
| 1-(3-hydroxy-4-methoxyphenyl)-4-methyl-5- | 8 | 6 | 1.88 ± 0.35* | 1.94 ± 0.22* |
| ethyl-7,8-dimethoxy-5H-2,3-benzodiazepine | | | | |
| (16 mg/kg IP daily) + DSS | | | | |

Statistically significant difference from vehicle + DSS control -
*p < 0.05;
**p < 0.01
DAI - Disease Activity Index;
DAIWT - DAI without weight loss parameter.

Table 5 Lists data for the colon length assessment for test animals in the DSS-induced colitis study.

TABLE 5

| | | | Mean colon | % of | Colon |
|---|---|---|---|---|---|
| | | | length | normal | shortening |
| Test substance | n | Group | CM ± SEM | length | inhib. % |
| Vehicle IP daily + water | 10 | 1 | 12.7 ± 0.15** | 100 | — |
| Vehicle IP daily + DSS | 8 | 2 | 7.1 ± 0.16 | 56.3 | — |
| Sulfasalazine | 7 | 3 | 8.8 ± 0.29** | 69.5 | 30 |
| (300 mg/kg IP daily) + DSS | | | | | |
| 1-(3-hydroxy-4- | 7 | 4 | 9.2 ± 0.33** | 72.5 | 37 |
| methoxyphenyl)-4-methyl-5- | | | | | |
| ethyl-7,8-dimethoxy-5H-2,3- | | | | | |
| benzodiazepine | | | | | |
| (64 mg/kg IP daily) + DSS | | | | | |
| 1-(3-hydroxy-4- | 8 | 5 | 7.9 ± 0.08 | 62.3 | 14 |
| methoxyphenyl)-4-methyl-5- | | | | | |
| ethyl-7,8-dimethoxy-5H-2,3- | | | | | |
| benzodiazepine | | | | | |
| (32 mg/kg IP daily) + DSS | | | | | |
| 1-(3-hydroxy-4- | 8 | 6 | 8.9 ± 0.40** | 70.2 | 32 |
| methoxyphenyl)-4-methyl-5- | | | | | |
| ethyl-7,8-dimethoxy-5H-2,3- | | | | | |
| benzodiazepine | | | | | |
| (16 mg/kg IP daily) + DSS | | | | | |

Significant difference from vehicle + DSS control −
*p < 0.05;
**p < 0.01
One way ANOVA and Dunnett Multiple Comparison Test: Control = Vehicle + DSS.

Table 6 lists the percent weight change for the test animals in the DSS-induced colitis study.

TABLE 6

| | | Mean weight (g) and % weight change ± SEM | | | | | | |
|---|---|---|---|---|---|---|---|---|
| Group | Test Substance | Day 0 | Day 7 | % change | Day 8 | % change | Day 9 | % change |
| 1 | Vehicle IP daily + water | 24.4 ± 0.2 | 25.3 ± 0.3 | +3.7 ± 1.0 | 25.4 ± 0.4¹ | 4.1 ± 0.01 | 26.1 ± 0.4 | +6.9 ± 0.8¹ |
| 2 | Vehicle IP daily + DSS | 23.3 ± 0.2 | 22.2 ± 0.2 | −5.4 ± 1.6 | 20.8 ± 0.6 | −10.8 ± 2.5 | 19.3 ± 0.8 | −17.2 ± 3.4 |
| 3 | Sulfasalazine | 22.6 ± 0.2 | 22.4 ± 0.2 | −0.6 ± 0.5 | 20.0 ± 0.4 | −11.4 ± 1.4 | 18.6 ± 0.4 | −17.7 ± 1.8 |
| | (300 mg/kg IP daily) + DSS | | | | | | | |
| 4 | 1-(3-hydroxy-4- | 23.7 ± 0.2 | 23.0 ± 0.6 | −2.7 ± 2.8 | 22.5 ± 0.9 | −4.8 ± 4.3 | 21.3 ± 1.1 | −9.7 ± 4.9 |
| | methoxy-phenyl)-4- | | | | | | | |
| | methyl-5-ethyl-7,8- | | | | | | | |
| | dimethoxy-5H-2,3- | | | | | | | |
| | benzodiazepine | | | | | | | |
| | (64 mg/kg IP daily) + DSS | | | | | | | |
| 5 | 1-(3-hydroxy-4- | 24.8 ± 0.2 | 24.1 ± 0.5 | −2.5 ± 1.7 | 22.5 ± 0.7 | −9.1 ± 2.6 | 20.9 ± 1.0 | −15.7 ± 4.1 |
| | methoxy-phenyl)-4- | | | | | | | |
| | methyl-5-ethyl-7,8- | | | | | | | |
| | dimethoxy-5H-2,3- | | | | | | | |
| | benzodiazepine | | | | | | | |
| | (32 mg/kg IP daily) + DSS | | | | | | | |
| 6 | 1-(3-hydroxy-4- | 24.6 ± 0.5 | 24.8 ± 0.8 | +0.5 ± 2.2 | 24.3 ± 1.0 | −1.7 ± 3.0 | 22.9 ± 1.4 | −7.5 ± 4.3 |
| | methoxy-phenyl)-4- | | | | | | | |
| | methyl-5-ethyl-7,8- | | | | | | | |
| | dimethoxy-5H-2,3- | | | | | | | |
| | benzodiazepine | | | | | | | |
| | (16 mg/kg IP daily) + DSS | | | | | | | |

¹Significant difference from Vehicle + DSS group - p < 0.01 - One way ANOVA and Dunnett Multiple Comparisons Test: Control = Vehicle + DSS

The data show that at a dose of 16 mg per kg, 1-(3-hydroxy-4-methoxyphenyl)-4-methyl-5-ethyl-7,8-dimethoxy-5H-2,3-benzodiazepine provided significant reduction in the DAI and DAIWT scores. Higher doses did not produce a significant decrease. The data also show that 1-(3-hydroxy-4-methoxy-phenyl)-4-methyl-5-ethyl-7,8-dimethoxy-5H-2,3-benzodiazepine, at doses of 64 mg/kg and 16 mg/kg, significantly inhibited colonic shortening. The inhibition of colonic shortening was greatest (37%) at the 64 mg/kg dose.

There was no observed significant difference in weight loss as compared to the negative controls. However, weight loss at both Day 8 and Day 9 were less than that observed in the DSS+vehicle group.

Sulfasalazine, dosed at 300 mg/kg daily, did not produce a statistically significant decrease in the DAI or DAINWT scores. There was no observed significant difference in weight loss as compared to the negative controls.

All references cited herein are incorporated by reference. The present invention may be embodied in other specific forms without departing from the spirit or essential attributes thereof and, accordingly, reference should be made to the appended claims, rather than to the foregoing specification, as indication the scope of the invention.

What is claimed is:

1. A pharmaceutical composition comprising a pharmaceutically acceptable carrier and 1-(3-hydroxy-4-methoxyphenyl)-4-methyl-5-ethyl-7,8-dimethoxy-5H-2,3-benzodiazepine, or a pharmaceutically acceptable salt thereof.

2. The pharmaceutical composition according to claim 1, comprising (R)-1-(3-hydroxy-4-methoxyphenyl)-4-methyl-5-ethyl-7,8-dimethoxy-5H-2,3-benzodiazepine substantially free of the corresponding (S)-enantiomer, or a pharmaceutically acceptable salt thereof.

3. The composition according to claim 2 wherein the amount of (R)-1-(3-hydroxy-4-methoxyphenyl)-4-methyl-5-ethyl-7,8-dimethoxy-5H-2,3-benzodiazepine, or pharmaceutically acceptable salt thereof, in the composition is 85% by weight or more of the total weight of 1-(3-hydroxy-4-methoxyphenyl)-4-methyl-5-ethyl-7,8-dimethoxy-5H-2,3-benzodiazepine.

4. The composition according to claim 3 wherein the amount of (R)-1-(3-hydroxy-4-methoxyphenyl)-4-methyl-5-ethyl-7,8-dimethoxy-5H-2,3-benzodiazepine, or pharmaceutically acceptable salt thereof, in the composition is 90% by weight or more of the total weight of 1-(3-hydroxy-4-methoxyphenyl)-4-methyl-5-ethyl-7,8-dimethoxy-5H-2,3-benzodiazepine.

5. The composition according to claim 4 wherein the amount of (R)-1-(3-hydroxy-4-methoxyphenyl)-4-methyl-5-ethyl-7,8-dimethoxy-5H-2,3-benzodiazepine, or pharmaceutically acceptable salt thereof, in the composition is 95% by weight or more of the total weight of 1-(3-hydroxy-4-methoxyphenyl)-4-methyl-5-ethyl-7,8-dimethoxy-5H-2,3-benzodiazepine.

6. The composition according to claim 5 wherein the amount of (R)-1-(3-hydroxy-4-methoxyphenyl)-4-methyl-5-ethyl-7,8-dimethoxy-5H-2,3-benzodiazepine, or pharmaceutically acceptable salt thereof, in the composition is 99% by weight or more of the total weight of 1-(3-hydroxy-4-methoxyphenyl)-4-methyl-5-ethyl-7,8-dimethoxy-5H-2,3-benzodiazepine.

7. The pharmaceutical composition according to claim 1, comprising (S)-1-(3-hydroxy-4-methoxyphenyl)-4-methyl-5-ethyl-7,8-dimethoxy-5H-2,3-benzodiazepine substantially free of the corresponding (R)-enantiomer, or a pharmaceutically acceptable salt thereof.

8. The composition according to claim 7 wherein the amount of (S)-1-(3-hydroxy-4-methoxyphenyl)-4-methyl-5-ethyl-7,8-dimethoxy-5H-2,3-benzodiazepine, or pharmaceutically acceptable salt thereof, in the composition is 85% by weight or more of the total weight of 1-(3-hydroxy-4-methoxyphenyl)-4-methyl-5-ethyl-7,8-dimethoxy-5H-2,3-benzodiazepine.

9. The composition according to claim 8 wherein the amount of (S)-1-(3-hydroxy-4-methoxyphenyl)-4-methyl-5-ethyl-7,8-dimethoxy-5H-2,3-benzodiazepine, or pharmaceutically acceptable salt thereof, in the composition is 90% by weight or more of the total weight of 1-(3-hydroxy-4-methoxyphenyl)-4-methyl-5-ethyl-7,8-dimethoxy-5H-2,3-benzodiazepine.

10. The composition according to claim 9 wherein the amount of (S)-1-(3-hydroxy-4-methoxyphenyl)-4-methyl-5-ethyl-7,8-dimethoxy-5H-2,3-benzodiazepine, or pharmaceutically acceptable salt thereof, in the composition is 95% by weight or more of the total weight of 1-(3-hydroxy-4-methoxyphenyl)-4-methyl-5-ethyl-7,8-dimethoxy-5H-2,3-benzodiazepine.

11. The composition according to claim 10 wherein the amount of (S)-1-(3-hydroxy-4-methoxyphenyl)-4-methyl-5-ethyl-7,8-dimethoxy-5H-2,3-benzodiazepine, or pharmaceutically acceptable salt thereof, in the composition is 99% by weight or more of the total weight of 1-(3-hydroxy-4-methoxyphenyl)-4-methyl-5-ethyl-7,8-dimethoxy-5H-2,3-benzodiazepine.

12. The pharmaceutical composition according to claim 1, comprising a racemic mixture of (R)- and (S)-enantiomers of 1-(3-hydroxy-4-methoxyphenyl)-4-methyl-5-ethyl-7,8-dimethoxy-5H-2,3-benzodiazepine, or a pharmaceutically acceptable salt thereof.

13. A method of treating an LTB₄-mediated inflammatory disorder in an individual in need of such treatment, comprising administering to said individual a therapeutically effective amount of the composition according to claim 1.

14. A method of treating an LTB₄-mediated inflammatory disorder in an individual in need of such treatment, comprising administering to said individual a therapeutically effective amount of the composition according to claim 2.

15. A method of treating an LTB₄-mediated inflammatory disorder in an individual in need of such treatment, comprising administering to said individual a therapeutically effective amount of the composition according to claim 7.

16. A method of treating an LTB₄-mediated inflammatory disorder in an individual in need of such treatment, comprising administering to said individual a therapeutically effective amount of the composition according to claim 12.

17. The method of claim 13 wherein the disorder is inflammatory bowel disease.

18. The method of claim 13 wherein the disorder is ulcerative colitis.

19. The method of claim 13 wherein the disorder is psoriasis.

20. The method of claim 13 wherein the disorder is rheumatoid arthritis.

21. The method of claim 13 wherein the disorder is Crohn's Disease.

22. The method of claim 13 wherein the disorder is radiation-induced gastrointestinal inflammation.

23. A method of preventing or delaying the onset of an inflammatory disorder mediated by LTB₄ in an individual who is at risk of developing an inflammatory disease state, said method comprising administering to said individual a therapeutically effective amount of the composition according to claim 1.

24. A method of preventing or delaying the onset of an inflammatory disorder mediated by LTB₄ in an individual who is at risk of developing an inflammatory disease state, said method comprising administering to said individual a therapeutically effective amount of the composition according to claim 2.

25. A method of preventing or delaying the onset of an inflammatory disorder mediated by LTB₄ in an individual who is at risk of developing an inflammatory disease state, said method comprising administering to said individual a therapeutically effective amount of the composition according to claim 7.

26. A method of preventing or delaying the onset of an inflammatory disorder mediated by LTB₄ in an individual who is at risk of developing an inflammatory disease state, said method comprising administering to said individual a therapeutically effective amount of the composition according to claim 12.

27. A method of treating an TXA₂-mediated disorder in an individual in need of such treatment, comprising administering to said individual a therapeutically effective amount of the composition according to claim 1.

28. A method of treating an TXA₂-mediated disorder in an individual in need of such treatment, comprising administering to said individual a therapeutically effective amount of the composition according to claim 2.

29. A method of treating an TXA₂-mediated disorder in an individual in need of such treatment, comprising administering to said individual a therapeutically effective amount of the composition according to claim 7.

30. A method of treating an TXA₂-mediated disorder in an individual in need of such treatment, comprising administering to said individual a therapeutically effective amount of the composition according to claim 12.

31. The method according to claim 27 wherein the TXA₂-mediated disorder comprises a chronic inflammatory disorder.

32. The method according to claim 31, wherein the chronic inflammatory disorder is selected from the group consisting of chronic fatigue syndrome/fibromyalgia, infertility, osteonecrosis of the jaw, multiple sclerosis, depression, autism, Crohn's Disease, Inflammatory Bowel Disease, late Lyme Disease, Sjogren's Syndrome, transient ischemic attack, attention deficit disorder and Parkinson's Disease.

33. The method of claim 27 wherein the disorder mediated by thromboxane A₂ involves an immune system activation of coagulation.

34. The method according to claim 27 wherein the disorder mediated by thromboxane A₂ is pain.

35. The method according to claim 27 wherein the disorder mediated by thromboxane A₂ is asthma.

36. The method according to claim 27 wherein the disorder mediated by thromboxane A₂ is angiogenesis associated with a developing tumor.

37. A method of preventing or delaying the onset of an inflammatory disorder mediated by TXA₂ in an individual who is at risk of developing an inflammatory disease state, said method comprising administering to said individual a therapeutically effective amount of the composition according to claim 1.

38. A method of preventing or delaying the onset of an inflammatory disorder mediated by TXA₂ in an individual who is at risk of developing an inflammatory disease state, said method comprising administering to said individual a therapeutically effective amount of the composition according to claim 2.

39. A method of preventing or delaying the onset of an inflammatory disorder mediated by TXA₂ in an individual who is at risk of developing an inflammatory disease state, said method comprising administering to said individual a therapeutically effective amount of the composition according to claim 7.

40. A method of preventing or delaying the onset of an inflammatory disorder mediated by TXA₂ in an individual who is at risk of developing an inflammatory disease state, said method comprising administering to said individual a therapeutically effective amount of the composition according to claim 12.
